(19) United States
(12) Patent Application Publication
(10) Pub. No.: US 2016/0272506 A1
(43) Pub. Date: Sep. 22, 2016

(54) METHOD OF AND A SYSTEM FOR DETERMINING A QUALITY PARAMETER IN AN AQUEOUS FLUID AND A METHOD OF CONTROLLING A QUALITY PARAMETER

(71) Applicant: NANONORD A/S, Alborg (DK)

(72) Inventor: Ole Norgaard Jensen, Alborg (DK)

(21) Appl. No.: 15/036,093

(22) PCT Filed: Nov. 13, 2014
(86) PCT No.: PCT/DK2014/050383

§ 371 Date: May 12, 2016

(30) Foreign Application Priority Data
Nov. 13, 2013 (DK) PA 2013 70682

(51) Int. Cl. C02F 1/00; G01N 33/18; B01D 63/10; B01D 61/02; B01D 61/14; B01D 63/08; G01N 24/08; C02F 1/44

(52) U.S. Cl. CPC C02F 1/008; G01N 24/084; G01N 33/1813; G01N 33/1833; G01N 33/1846; C02F 1/441; C02F 1/444; C02F 1/442; B01D 61/025; B01D 61/027; B01D 61/145; B01D 63/08; B01D 63/10; C02F 2101/20

(57) ABSTRACT

The invention concerns a system for and a method of determining a least one quality parameter in an aqueous fluid. The method including subjecting at least a sample of the aqueous fluid to a cross-flow filtration in a cross-flow filter, separating the aqueous fluid into a permeate fraction and a retentate fraction, performing NMR reading on the retentate fraction using an NMR spectroscope, collecting NMR data from said NMR reading and correlating the collected NMR data to calibration data to determine said at least one quality parameter of the aqueous fluid.

### TECHNICAL FIELD

The invention relates to a method and a system for determining a quality parameter in an aqueous fluid, such as waste water, lake water and other aqueous fluids where quality is often important as well as a method of performing a water cleaning process.

### BACKGROUND ART

Quality parameters in aqueous fluids such as waste water, drinking water, ground and surface water are today determined using different methods. A standard method of determination of common inorganic anions in environmental waters in the US is for example the use of ion chromatography.

Such methods generally requires the use of large and expensive ion chromatographs and are generally very time consuming and labor consuming to perform.

Also laboratory analysis of the compounds in waste water by gas chromatography or mass spectrometry (GC/MS) is often applied for determining of a quality parameter of an aqueous fluid.

Very often the component(s) that is/are required to be determined to establish the quality parameter is/are present in very small amounts which makes any quantitative determinations very difficult, expensive and/or time consuming and often the determinations are rather inaccurate.

### DISCLOSURE OF INVENTION

An object of the invention is to provide a new method for determining a quality parameter in an aqueous fluid which method is relatively fast and where the quality parameter can be determined with a very high accuracy.

Another object of the invention is to provide a system for determining a quality parameter in an aqueous fluid which system is relatively fast and where the quality parameter can be determined with a very high accuracy. Further it is desired that the system can be employed for determination of several different quality parameters.

An additional object of the invention is to provide a new method of performing a water cleaning process by using the method of determining the quality parameter

These objects have been solved by the present invention as defined in the claims.

The method or the system of the invention for determining the quality parameter as well as the method of performing a water cleaning process have shown to have a large number of advantages which will be clear from the following description.

It should be emphasized that the term “comprises/comprising” when used herein is to be interpreted as an open term, i.e. it should be taken to specify the presence of specifically stated feature(s), such as element(s), unit(s), integer(s), step(s) component(s) and combination(s) thereof, but does not preclude the presence or addition of one or more other stated features.

Reference made to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the skilled person will understand that particular features, structures, or characteristics may be combined in any suitable manner within the scope of the invention as defined by the claims.

The term “substantially” should herein be taken to mean that ordinary product variances and tolerances are comprised.

Reference made to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the skilled person will understand that particular features, structures, or characteristics may be combined in any suitable manner within the scope of the invention as defined by the claims.

The method of the invention for determining a least one quality parameter in an aqueous fluid has been found to be surprisingly fast and accurate and simultaneously the quality parameter may comprise concentration of one or more components which are present in low amounts such as less than 100 ppm or even less than 10 ppm or 1 ppm (1 ppm=1 mg/I of the aqueous fluid). The method comprises subjecting at least a sample of the aqueous fluid to a cross-flow filtration in a cross-flow filter, separating the aqueous fluid into a permeate fraction and a retentate fraction and thereafter performing NMR reading on the retentate fraction using an NMR spectroscope, collecting NMR data from the NMR reading and correlating the collected NMR data to calibration data to determine the at least one quality parameter of the aqueous fluid.

Nuclear magnetic resonance—abbreviated NMR—is a phenomenon which occurs when the nuclei of an isotope in a magnetic field absorb and re-emit electromagnetic radiation. The emitted electromagnetic radiation has a specific resonance frequency which depends on the strength of the magnetic field and the magnetic properties of the isotope. NMR allows the observation of specific quantum mechanical magnetic properties of the atomic nucleus. Many scientific techniques exploit NMR phenomena to study molecular physics, crystals, and non-crystalline materials through NMR spectroscopy. NMR is also routinely used in advanced medical imaging techniques, such as in magnetic resonance imaging (MRI).

NMR measurement is performed by NMR spectroscopy and comprises using the NMR phenomenon to study materials e.g. for analyzing organic chemical structures. NMR spectroscopy is well known in the art and has for many years been applied for laboratory measurements in particular where other measurement methods could not be used. NMR spectroscopy is performed using a NMR spectroscopy. Examples of spectrometer are e.g. described in U.S. Pat. No. 6,310,480 and in U.S. Pat. No. 5,023,551.

A spectrometer comprises a unit for providing a magnetic field e.g. a permanent magnet assembly as well as a transmitter and a receiver for transmitting and/or receiving RF frequency pulses/signals The RF receiver and RF transmitter are connected to an antenna or an array of RF antennae, which may be in the form of transceivers capable of both transmitting and receiving. The spectrometer further comprises at least one computing element, in the following referred to as a computer.

The intensity of nuclear magnetic resonance signals and, hence, the sensitivity of the technique depends on the strength of the magnetic field and generally the NMR spectrometer applied for quantitative determination should have relatively large magnets—often electro or permanent magnets. The smaller the magnetic field, the more noise and accordingly the more measurements and time of NMR reading is required to obtain a result of a desired accuracy.

According to the present invention it has been found that by subjecting the aqueous fluid sample to a cross-flow filtration to thereby separating the aqueous fluid into a permeate fraction and a retentate fraction and thereafter performing NMR reading on the retentate fraction using an NMR spectroscope a much faster determination of a quality parameter can be obtained or in the alternative a lower magnetic field can be used for performing the NMR reading to obtain a determination of a desired accuracy of the at least one quality parameter of the aqueous fluid.

General background of NMR formation evaluation can be found, for example in U.S. Pat. No. 5,023,551.

A general background description of NMR measurement can be found in “NMR Logging Principles and Applications” by George R. Coates et al, Halliburton Energy Services, 1999. See in particular chapter 4.

Although ‘NMR reading’ in the following often will be used in singular to describe the invention, it should be observed that the singular term ‘NMR reading’ also includes a plurality of NMR readings unless other is specified. NMR reading means performing NMR spectroscopy on the sample in question.

The terms ‘NMR reading’ and ‘NMR Measurement’ are used interchangeable. The phrase “NMR accumulated reading time” means the total time for performing one or more NMR readings to obtain NMR data for quantitative determination of at least one isotope to determine the at least one quality parameter of the aqueous fluid.

Cross-flow filtration (sometimes called tangential flow filtration) is a well know filtration method and is often used in industrial productions e.g. for liquid processing to effect clarification, product isolation, concentration and/or separation in a large number of manufacturing industries.

In cross-flow filtration, an incoming feed stream passes across the surface of a cross-flow membrane, and two exiting streams are generated. The permeate stream is the portion of the fluid that passes through the membrane. This filtered fluid will contain some percentage of soluble and/or insoluble components from the initial feed stream that are smaller than the membrane removal rating. The remainder of the feed stream, which does not pass through the cross-flow membrane, is known as the retentate stream (sometimes called the concentrate stream). Advantageously the cross-flow filtration is a microfiltration (MF), an ultrafiltration (UF), a nanofiltration (NF) and/or a reverse osmosis (RO).

Microfiltration is a low-pressure process for the retention of suspended material particle size of 0.01 microns or larger. Smaller particles (salts, sugars and proteins, for example) pass through the membrane. Typical operating pressure (pressure difference over the membrane) is up to about 3 bars. Microfiltration membranes have pore sizes larger than about 0.1 μm.

Ultrafiltration is a medium-pressure process offering retention of proteins, colloids and biological material including particles 0.005 microns or larger (molecular weight greater than 1000 Dalton). Typical operating pressure ranges from about 0.48 to about 10 bars. Ultrafiltration membranes have pore sizes ranging from about 0.1 μm to about 0.01 μm

In nanofiltration water and monovalent ions, as well as low molecular weight substances (less than 250 Dalton) pass through nanofiltration membranes. Divalent or multivalent ions, such as divalent salts, are retained. Operating pressure up to about 40 bars is typical. Nanofiltration membranes have pores sized from about 0.001 μm to about 0.01 μm, smaller than that used in microfiltration and ultrafiltration, but just larger than that in reverse osmosis.

Reverse Osmosis is known as a relatively high-pressure process that retains almost all particles and ionic species, while water and some organic molecules pass through. Substances with molecular weight above 50 Dalton are preferably retained almost without exception. In prior art RO and NF procedure it is normally desired to have a very high operation pressure and a high flux. In principle the operation pressure can be as high as desired e.g. up to about 60 bars, however in the present invention operation pressure of from about 4 bars and higher has been found to be suitably. Generally the higher the operation pressure the faster the separation will be completed. However, higher operating pressure result in higher cost and it has been found that operation pressure of about 5 to about 10 bars are preferred and in particular operation pressure from about 8 to about 10 bars giver well performing and economically feasible solutions. However since the aqueous fluid sample is normally relatively small compared to when RO is applied in a production process it has been found that even where the operation pressure is relatively low the total time for performing the determination of the quality parameter can be reduced significantly compared to corresponding determinations without the cross-flow filtration.

It has been found that in general total time (the NMR accumulated reading time) for performing the required NMR readings for quantitative determination of an isotope or a component comprising such isotope with a desired signal to noise follows the concentrations to the second power. In other words a 10 fold increase in concentration result in a factor 100 reduced NMR reading time. Alternatively or simultaneously the signal to noise level can be substantially increased.

When performing the determination of the quality parameter based on the NMR data obtained from the NMR reading on the retentate fraction it is required to know or have an estimation of the relative size of the retentate fraction relative to the aqueous fluid sample. This can be obtained by a direct measurement of the amount of (preferably weight (mass) or volume) at least two of the retentate fraction, the permeate fraction and the aqueous fluid sample. Alternatively or in combination the amount of one or two of the retentate fraction, the permeate fraction and the aqueous fluid sample can be estimated base on filtration time and pressure and/or flow. The skilled person will be able to find a suitable way of determining the relative size of the retentate fraction relative to the aqueous fluid sample.

In an embodiment the method comprises withdrawing the sample of the aqueous fluid, performing the cross-flow filtration, performing the NMR reading and determine the at least one quality parameter of the aqueous fluid.

By withdrawing a sample of a predetermined size a good control of the size of the aqueous fluid sample is obtained. To find the size of the retentate fraction the flow of the permeate may advantageously be determined or the flow of the retentate fraction may e.g. be determined e.g. when discharging the retentate fraction after the NMR reading has been performed.

In an embodiment the method comprises performing the cross-flow filtration and the NMR reading in-line on the retentate fraction. This can be done by flowing the retentate fraction directly from the cross-flow filtration to the NMR spectroscope for performing the NMR reading. The aqueous fluid sample may e.g. be withdrawn from the total aqueous fluid prior to performing the cross-flow filtration and the NMR reading in-line on the aqueous fluid sample or the aqueous fluid sample may flowed directly from the aqueous fluid to the cross-flow filtration. In the latter situation the size of the aqueous fluid sample may e.g. be obtained by determining the flow of the aqueous fluid sample.

The NMR reading is advantageously performed on the retentate fraction in flowing condition or in semi flowing condition.

Where the NMR reading is performed on the on the retentate fraction in flowing condition the method may advantageously comprise determination of the flow of the retentate fraction in the magnet field.

The phrase that the NMR reading is performed on the retentate fraction in flowing condition means that the retentate fraction is flowing through the magnetic field during the reading.

The phrase that the NMR reading is performed on the retentate fraction in semi flowing condition means that the retentate fraction is flowing through the magnetic field and temporarily stopped during at least a part of the reading.

When performing the NMR measurement on the retentate fraction in flowing condition it should advantageously be ensured that the velocity of the flowing retentate fraction is adjusted or kept such that the retentate fraction is within the spectrometer range for a sufficient time to perform the NMR measurement.

In an embodiment the NMR reading is performed on the retentate fraction in flowing condition or in semi flowing condition.

The method advantageously comprises subjecting the aqueous fluid to the cross-flow filtration and flowing at least a part of the retentate fraction to a magnetic field of the NMR spectroscope and performing the NMR reading.

Where the cross-flow filter is selected such that not all the isotopes to be determined is retained in the retentate fraction, the NMR data is calibrated to compensate for the isotopes that has passed to the permeate.

In an embodiment the cross-flow filter is selected such that an isotope bound in a relatively large compound is retained in the retentate fraction, whereas the same isotope in smaller compounds or in ionic form is passes to the permeate. Thereby determination of the isotope bound to the larger compound may in a simple way be determined.

Advantageously the cross-flow filtration is adjusted such that the permeate fraction is larger than the retentate fraction. Thereby a suitable high flux of fluid can be obtained. The “flux” is the rate of sample flow through the membrane—i.e. the rate of the permeate, measured in volume/unit time

To obtain a suitable flux the membrane or membranes of the cross-flow filter is advantageously selected in dependence on the impurities and impurity concentration of the aqueous fluid. Preferably the permeate fraction is up to about 99.9 vol %, such as from about 50 to about 99 vol %, such as from about 60 to about 95 vol %, of the total aqueous fluid sample. In an embodiment the final permeate fraction—i.e. after optional recirculation is terminated—is up to about 99.9 vol %, such as from about 50 to about 99 vol %, such as from about 60 to about 95 vol %, of the total aqueous fluid sample.

Advantageously the method comprises determining the relative mass or volume of the retentate fraction relative to mass or volume of at least one of the sample or the permeate.

The determination of volume/mass can be performed by measurement, by calculation (e.g. based on pressure difference over membrane, membrane area and filter time), or by estimation (base on e.g. one parameter such as filter time and calibrated with earlier determinations).

The cross-flow filter can in principle be any kind of cross-flow filter comprising at least one membrane for the cross-flow filtration. The cross-flow filter is often defined in relation to the type of membrane used and may advantageously comprise a MF membrane, an UF membrane, a NF membrane a RO membrane or two or more of these in any suitable combination.

The membrane may be a ceramic membrane, a metal membrane, a polymer membrane or a composite membrane comprises two or more of the before mentioned materials

In an embodiment the cross-flow filter is a ceramic filter comprising a ceramic filter membrane. Such a membrane is for example described in U.S. Pat. No. 7,699,903 describes a ceramic cross-flow filter comprising a multi layered SiC ceramic filter body for cross-flow filtration.

In an embodiment the cross-flow filter comprises a thin-film composite membrane (TFC), such as a TFC comprising two or more layers. In an embodiment the TFC membrane comprises a thin polyamide layer (<200 nm) deposited on top of a polyethersulfone or polysulfone porous layer (about 50 microns) optionally on top of a substrate such as a non-woven fabric support sheet.

In an embodiment the cross-flow filter comprises a polymer membrane, preferably comprising at least one layer of PVDF, polyamide, cellulose acetate, Polypiperazine amide Polyamide-urea, Polyethersulfone and mixtures thereof. The polymer membrane may e.g. comprise a metal layer—e.g. steel layer for support.

Other materials and combinations of materials which are usually applied for cross-flow filtration may also be applied in the present invention.

The shape of the membrane may e.g. be a tubular design, a hollow design, a spiral wound design or a flat sheet design. Such designs are well known in the

In a preferred embodiment the cross-flow filter comprises a flat sheet membrane optionally placed on a support material. This solution is very simple and allows easy replacement of the membrane.

In an embodiment the cross-flow filter comprises a coiled membrane (spiral membrane) such as a spiral-wound membrane module. A spiral membrane is usually composed of a combination of flat membrane sheets separated by a thin meshed spacer material which serves as a porous plastic screen support. These sheets are rolled around a central perforated tube and fitted into a tubular steel pressure vessel casing. The feed solution passes over the membrane surface and the permeate spirals into the central collection tube. Spiral-wound membrane modules are very compact and relatively cheap.

Advantageously the cross-flow filter is a reverse osmosis filter and the cross-flow filtration is or comprises reverse osmosis. The cross-flow filter may e.g. comprise a MF membrane, a UF membrane and/or a NF membrane as pre-filter membrane and a RO membrane. In a preferred embodiment the cross-flow filter comprises a MF membrane and OR filter, where the MF membrane is used as pre-filter

Advantageously the method comprises recirculating the retentate fraction in the cross-flow filter followed by performing NMR reading on the retentate fraction. Where the cross-flow filter comprises one or more pre-filter membrane(s) the recirculation is advantageously not recirculated in such pre-filter membrane(s) but only in the final membrane with the smallest pore size.

In an embodiment the method comprises recirculating in a closed loop, the method comprising withdrawing the aqueous fluid sample and subjecting the aqueous fluid sample to the cross-flow filtration in a recirculating loop comprising recirculating the retentate fraction for additional filtration.

The recirculation may be continued for a preselected time interval or until a preselected amount of permeate fraction has been obtained.

In an embodiment the method comprises feeding the aqueous fluid sample in a stream to the cross-flow filter for cross-flow filtration and recirculating the retentate fraction for additional filtration together with the stream of the aqueous fluid sample at least until the entire aqueous fluid sample has passes the cross-flow filter. If desired the recirculation may be continued e.g. for a predetermined time.

In an embodiment the retentate fraction is recirculated for a predetermined time, such as for 1 minute or more, such as for 10 minutes or more, such as for 1 hour or more, such as up to 24 hours.

The time of recirculation depend largely on the cross-flow filtration used, the quality parameter to be determined and on the purity of the aqueous fluid with respect to the one or more isotopes or components that are relevant for the quality parameter.

In an embodiment the retentate fraction is recirculated in up to 8 hours, such as from about 10 minutes to about 5 hours.

When the final retentate fraction has been obtained it is subjected to the NMR reading and advantageously a new aqueous fluid sample is subjected to the cross-flow filtration with recirculation in the cross-flow filter.

In an embodiment the retentate fraction is recirculated to obtain a predetermined retentate fraction size, such as from about 1 ml to about 10 l, such as from about 5 ml to about 2 l, such as from about 10 ml to about 0.5 l. The final retentate fraction is advantageously a fraction of about 1 to about 50% of the aqueous fluid sample, such as from about 2 to about 10% of the aqueous fluid sample, such as from about 3-6% of the aqueous fluid sample.

As mentioned above the method advantageous comprises performing a plurality of NMR readings in order to reduce noise and obtain a desired precision.

In an embodiment the at least one NMR reading comprises a reading at least one NMR readable isotope. Preferably the reading comprises a reading a plurality of NMR, readable isotopes. Thereby one or more quality parameter may be determiner very fast.

The NMR reading may in principle comprise NMR reading of any NMR readable isotopes

In an embodiment the method comprises NMR reading of one or more of the isotopes ¹H, ¹⁰B, ¹¹B, ¹³C, ¹⁴N, ¹⁵N ¹⁹F ²³Na, ²⁷Al, ²⁹Si ³¹P, ³³S, ³⁵Cl, ³⁷Cl, and ³⁹K, ⁴¹K, ⁴³Ca, ⁴⁷Ti, ⁴⁹Ti, ⁵⁰V, ⁵¹V, ⁵³Cr, ⁵⁵Mn, ⁵⁷Fe, ⁵⁹Co, ⁶¹Ni, ⁶³Cu, ⁶⁵Cu, ⁶⁷Zn, ⁶⁹Ga, ⁷¹Ga, ⁷⁵As, ⁷⁷Se, ⁷⁹Br, ⁸¹Br, ⁸³Kr, ⁸⁵Rb, ⁸⁷Rb, ⁸⁷Sr, ⁸⁹Y, ⁹¹Zr, ⁹³Nb, ⁹⁵Mo, ⁹⁷Mo, ¹⁰⁵Pd, ¹⁰⁷Ag, ¹⁰⁹Ag, ¹¹¹Cd, ¹¹³Cd, ¹¹⁷Sn, ¹¹⁹Sn, ¹¹⁵Sn, ¹²¹Sb, ¹³⁵Ba, ¹³⁷Ba ¹⁷⁷Pb, ¹⁹⁹Hg, ²⁰¹Hg, ²⁰⁷Pb. Preferably the method comprises a plurality of readings of one or more of ¹³C, ¹⁴N, ¹⁹F ²³NA, ³¹P, ³⁵Cl, ³⁷Cl, ³⁹K, ⁷⁹Br, and ⁸¹Br.

When performing NMR reading on two or more isotopes it has been found that the NMR reading can be performed simultaneously or timely overlapping. For example the T1 or T2 times for reading one isotope need not be terminated prior to initiating the NMR reading including T1 and/or T2 time(s) for another isotope. Thereby the NMR reading of several isotopes may be performed relatively fast.

In an embodiment the method comprises NMR reading of one or more heavy metal isotopes, such as isotopes of Pb, Hg and/or Cd.

In an embodiment the method comprises a plurality of consecutive NMR readings of one or more NMR readable isotope preferably comprising at least one of ¹³C, ¹⁴N, ¹⁹F ²³NA ³¹P, ³⁵Cl, ³⁹K, ⁷⁹Br, and ⁸¹Br.

The quality parameter advantageously requires at least one quantitative determination of an isotope or a compound comprising an isotope.

In an embodiment the method comprises NMR reading of ³⁵Cl and/or ³⁷Cl and qualitatively and/or quantitatively determine one or more trihalomethanes and/or free chlorine and/or total chlorine contents.

In an embodiment the method comprises NMR reading of ¹H and ¹³C and qualitatively and/or quantitatively determine one or more hydrocarbons such as Methane (gas) or heavier hydrocarbons such as PAH (polycyclic aromatic hydrocarbon) or any other hydrocarbons.

In an embodiment the method comprises repeating determination of the at least one quality parameter of the aqueous fluid.

It has been found that the present invention may be applied as a quality monitoring facility e.g. for monitoring at least one quality parameter in water, such as drinking water, waste water, industrial water, optionally cleaned offshore waste water, lake water, sea water e.t.c.

In an embodiment wherein the method comprises monitoring of the at least one quality parameter of the aqueous fluid, by determine the at least one quality parameter with predetermined interval. Preferably the method comprises monitoring of the at least one quality parameter of the aqueous fluid, by determine the at least one quality parameter with predetermined interval

In an embodiment the method comprises monitoring the at least one quality parameter of the aqueous fluid, by with the predetermined time interval withdrawing a sample, subjecting the sample to the cross-flow filtration, obtaining the retentate fraction, performing the NMR reading on the retentate fraction and determine the at least one quality parameter of the aqueous fluid.

The quality parameter can in principle be any quality parameter based on the present or amount of one or more isotopes and/or one or more compounds comprising an isotope

Examples of quality parameters comprises nitrogen content, flour content, chlorine content, content of free chlorine (HOCL, OCl⁻), content of ammonium, content of ammonia, content of nitrate, content of nitrite, content of potassium, content of phosphor, content of organic matter, content of organic solvents, such as benzene, content of heavy metal(s), content of trihalomethane, content of total carbons (TC), content of total organic carbon (TOC), content of selected hydrocarbons (e.g. methane or butane), or any combinations thereof.

Advantageously the at least one quality parameter of the aqueous fluid is determined by generating NMR data from the at least one NMR reading and correlating the NMR data calibration data and adjusting depending on the retentate fraction to permeate fraction size (volume or weight/mass).

In an embodiment method comprises providing calibration data of samples with known amount of the isotope(s) and or compound(s) on which the quality parameter is based. The calibration data advantageously constitutes a calibration map. The calibration map comprises the desired NMR data and optionally additionally data such as data relating to temperature(s), pH value(s) and or relative amounts of selected components in dependence of pH value and/or temperature.

The term ‘calibrating map’ is herein used to designate a collection of NMR data obtained of samples with known amounts of the isotope(s) and or compound(s) on which the quality parameter is based and optionally other data which can be used in the interpretation of NMR data. The calibration map may be in form of raw data, in form of drawings, in form of graphs, in form of formulas or any combinations thereof. Advantageously the calibration data is stored in the computer of the NMR system and used by the computer in the processing of measured NMR data.

In an embodiment the method comprises providing a control loop adjusting the cross-flow filtration such that to obtain a preselected flux through the cross-flow filter to become permeate, wherein the preselected percentage is up to about 99.9 vol %, such as from about 50 to about 99 vol %, such as from about 60 to about 95 vol % Preferably the cross-flow filtration is a reverse osmosis filtration and the method comprises controlling a reverse osmosis backpressure.

In an embodiment the method comprises performing NMR reading on an unfiltered sample of the aqueous fluid, preferably the NMR reading on the unfiltered sample comprises NMR reading of at least one isotope which is also read on the retentate fraction, preferably the NMR reading on the unfiltered sample and the NMR reading on the retentate fraction comprises reading of a plurality of common isotopes.

Optionally method comprises performing NMR reading on unfiltered sample of the aqueous fluid at predetermined interval.

In an embodiment the NMR reading on unfiltered sample of the aqueous fluid has an unfiltered sample NMR accumulated reading time and the reading on the retentate fraction has an accumulated retentate fraction reading time, wherein the retentate fraction accumulated reading time is shorter than the unfiltered sample NMR accumulated reading time, preferably the retentate fraction accumulated reading time is about 0.9 times or less than the unfiltered sample NMR accumulated reading time, such as 0.5 times or less, such as about 0.3 times or less, such as 0.1 times or less, such as 0.01 times or less.

In an embodiment the NMR reading on unfiltered sample of the aqueous fluid has an unfiltered sample NMR accumulated reading time and the NMR reading on the retentate fraction has an accumulated retentate fraction reading time which are substantially equal. It will be seen that the signal to noise of the NMR data obtained by the NMR reading on unfiltered sample is much smaller than the signal to noise of the NMR data obtained by NMR reading on the retentate fraction.

The term NMR accumulated reading time means the total time for the reading or readings to reach a result. As mentioned it is often required to have many NMR readings to reduce noise and to have a sufficiently or desired signal to noise level. The phrase ‘NMR time span’ and ‘NMR accumulated reading time’ are used interchangeable.

Advantageously the method comprises togging between NMR reading on unfiltered sample and NMR reading on the retentate fraction. Thereby an effective control of the accuracy of the determination can be obtained.

In an embodiment the method comprises tracing one or more NMR isotopes and determine the respective concentration of the one or more isotopes in both the aqueous fluid and the retentate using an NMR accumulated reading time which is than the normal (required) NMR time span, such as up to 10 time or up to 100 or even up to 10000 times longer than the required NMR time span to obtain a quantitative determination). Thereby an essentially noise free determination can be obtained.

In an embodiment the method comprises calibrating the cross-flow filtration performance based on the difference in NMR data of the retentate fraction NMR reading and NMR data of the unfiltered sample NMR reading, preferably the method comprises triggering an alarm if the cross-flow filtration performance reach a preset minimum performance level.

In an embodiment the method comprises determining a quality parameter comprising a quantitative determination of one or more nitrogen containing compounds in the aqueous fluid. This is performed by quantitatively determination of nitrogen present in form of one or more nitrogen containing compounds or ions thereof of in an aqueous fluid. The method comprising subjecting at least a part of the aqueous fluid to an NMR reading comprising generating a ¹⁴N data comprising a ¹⁴N NMR data spectra and correlating the ¹⁴N NMR data to calibration data.

In an embodiment the nitrogen determination is performed on at least a part of the retentate fraction. Preferably the nitrogen determination is performed on substantially all of the retentate fraction. In an embodiment substantially all of the nitrogen containing components having a molecular weight of 200 Da or less will remain in the retentate fraction, the quantitative determination of the nitrogen containing component(s) can thereby in a simple way be calculated. In practice it has been found that the NMR reading on the retentate fraction often results in an increased homogeneity of nitrogen containing compounds which means that for many applications it will be sufficient to performing the NMR reading on only a part of the retentate fraction.

In an embodiment the method comprises calibrating the RO system performance based on the difference in the ¹⁴N NMR data of an unfiltered portion and the retentate fraction taking account for the amount of aqueous fluid sample. The method advantageously comprises triggering an alarm if the RO system performance reaches a preset minimum performance level.

In an embodiment the method comprises calibrating the RO system performance based on the difference in the concentration of one or more measured NMR isotopes of an unfiltered portion and the retentate fraction. The method advantageously comprises triggering an alarm if the RO system performance reaches a preset minimum performance level.

In an embodiment the difference in the concentration of one or more measured NMR isotopes of an unfiltered portion and the retentate fraction is used to determine a concentration factor where the concentration factor is an estimate of the retentate fraction amount divided by the aqueous fluid sample amount and is determined by the isotope(s) concentration in the unfiltered portion divided by the isotope(s) concentration in the retentate fraction.

Advantageously the NMR measurement comprises simultaneously subjecting the retentate fraction to a magnetic field B, and a plurality of pulses of radio frequency energy E (RF pulses) and receiving relaxation signals from isotope in question.

After the radio frequency pulse or pulses has/have excited the nuclei, the nuclei will preferably be allowed to relaxation which will continue over a time called the acquisition time or relaxation time thereby preferably giving an NMR signal due to an oscillating voltage induced by the precession of the nuclear spin. This result in a decaying sine wave is termed free induction decay (FID) data. In an embodiment the relaxation signals comprises a free induction decay (FID) data.

Advantageously the pulse sequence called a cycle of pulse sequence is repeated a plurality of times in order to improve signal-to-noise (S/N), which increases as the square root of the number of cycles.

Advantageously the FID data is processed using methods well known in the art preferably including subjecting the FID data to a furrier transformation to provide a frequency domain spectrum also called the ppm band or spectral band. The frequency domain spectrum shows the intensity as a function of frequency where the frequency width per ppm depend on the spectrometer and the size of its magnetic field i.e. the higher Tesla the larger frequency bandwidth per ppm.

Generally prior art NMR spectrometers operates with a relative high magnetic field e.g. 10 or 15 Tesla or even higher in order to have a high sensitivity (signal to noise ratio scales with 2^{nd} power of the magnetic field) for example in connection with RF saddle coil. However in accordance with the present invention it has been found that a relatively low magnetic field e.g. with a closely coupled helical coil actually provides an even more accurate determination. By using such a relatively low magnetic field the NMR spectrometer becomes much cheaper and further the required size of the NMR spectrometer is highly reduced which makes is much simpler to e.g. use a transportable NMR spectrometer.

Also it has been found to be desired to us a NMR spectrometer with a relatively large measurement volume, such as at least about 1 ml, such as at least about 5 ml, such as at least about 20 ml.

In an embodiment the NMR spectrometer generates frequency domain spectra with a frequency width per ppm of about 300 Hz/ppm or less, such as about 200 Hz/ppm or less, preferably of about 100 Hz/ppm or less, more preferably of about 70 Hz/ppm or less or even about 35 Hz/ppm or less.

Advantageously the NMR measurement comprises simultaneously subjecting the sample to a magnetic field B, and an exciting RF pulse with frequencies selected to excite a nucleus of spin of at least a part of the isotope(s) in question. Preferably the exciting RF pulse span over a band width (span over a frequency range) which is sufficient to excite isotope(s) in question.

The exciting RF pulse advantageously provided by impressing a RF pulse or a train of pulses with a stationary or varying field band width (Hz) for a sufficient time to saturate the nuclei. The time of application of the pulse is called the pulse width (μs). Generally the higher the field band width the lower pulse width is required.

Further the higher the magnetic field the higher frequency range of the exciting RF pulse is required for fully excite the nuclei of the isotope(s) in question.

Advantageously the frequency range of the exciting RF pulse spans over up to about 20 KHz, such as up to about 10 KHz.

In an embodiment the NMR reading is performed in a magnetic field of up to about 25 Tesla, such as from about 0.3 Tesla to about 15 Tesla.

It has been found that the magnetic field B beneficially may be selected to be relatively low while a high resolution with low noise can be obtained. In an embodiment the NMR reading is performed in a magnetic field of up to about 2.5 Tesla, such as from about 0.3 Tesla to about 1.5 Tesla. Due to this relatively low magnetic field the equipment for performing the NMR reading can be kept at a surprisingly low cost while simultaneously a high signal to noise determination can be obtained in a relatively short NMR accumulated reading time.

In an embodiment the magnetic field is generated by a permanent magnet, such as a neodymium magnet. Since permanent magnets are generally not costly, this solution provides a low cost solution which for many applications may provide a sufficient low noise and highly reliable result.

In an embodiment the magnetic field is generated by an electromagnet, such as a solenoid magnet or other electromagnets which are usually applied in motors, generators, transformers, loudspeakers or similar equipment.

Electromagnets of high strength e.g. electromagnets that can be applied for generating a field for NMR applications are often relatively expensive compared with permanent magnets however, still much cheaper that magnets used in prior art high resolution NMR spectrometers. In an embodiment it may be beneficial to use an electromagnet arranged to be adjustable by adjusting the current in the coil of the electromagnet to a desired level.

In an embodiment the magnetic field is generated by a permanent magnet in combination with an electromagnet which advantageously is constructed for providing a pulsed magnetic field.

In an embodiment the NMR reading is performed in a pulsed magnetic field.

In an embodiment the NMR reading is performed in a pulsed magnetic field. By pulsing the magnetic field even more accurate determinations can be obtained because measurements at different field strength provides a tool for identifying noise which may accordingly be filtered of.

Advantageously the NMR reading is performed in a magnetic field with a standard deviation of the field over the sample volume of more than 10 ppm such as from about 100 ppm to 3000 ppm.

In an embodiment of the invention the magnetic field in the measuring zone, i.e. the part where the sample to be measured on is located when the NMR measurement is performed, is preferably relatively spatially homogeneous and relatively temporally constant. However, in general it is difficult to provide that the magnetic field in the measuring zone is entirely homogenous and further for most magnetic fields, the field strength might drift or vary over time due to aging of the magnet, movement of metal objects near the magnet, and temperature fluctuations. In the present invention it has been found that minor inhomogeneity's of the magnetic field has not practical negative effect and in fact it is believed that minor inhomogeneity's of the magnetic field may in fact add to improve the accuracy of the NMR measurement all though at present it cannot be fully explained.

Drift and variations over time can be dealt with by controlling temperature and/or by applying a field lock such as it is generally known in the art.

Spatial in homogeneities of the magnetic field can be corrected for by a simple calibration or alternatively or simultaneously such spatial in homogeneities can be adjusted for by shim coils such as it is also known in the art. Such shim coils may e.g. be adjusted by the computer to maximize the homogeneity of the magnetic field.

In an embodiment of the invention the method comprises performing a plurality of NMR readings at a selected magnetic field, preferably the magnetic field is kept substantially stationary during the plurality of NMR readings. The data of the plurality of NMR readings is averaged (to reduce noise) and based on the averaged NMR data the determination of the quality parameter is performed. The time for performing the plurality of NMR readings is as mentioned referred to as the NMR accumulated reading time.

In an embodiment the method of the invention comprises regulating the temperature e.g. by maintaining the temperature at a selected value.

In an embodiment the method comprises performing the NMR reading at a fixed temperature.

In an embodiment the method of the invention comprises determining the temperature.

In an embodiment the method of the invention comprises performing the NMR readings at pulsed temperature.

In an embodiment the method comprises performing the NMR reading at temperature which is pulsed, preferably the pulsing range is from about 1° C. to about 90° C., such as from about 10° C. to about 80° C., such as from about 20° C. to about 70° C. The pulsed temperature may advantageously be applied for correlation of resulting measurements at different temperatures to eliminate errors and/or for improved pH determination as described above.

In an embodiment the radio frequency pulses are in form of adiabatic RF pulses, i.e. RF pulses that are amplitude and frequency modulated pulses.

In an embodiment the method comprises subjecting the sample to pulsed trains of RF pulses, preferably with repetition rates of at about 400 ms or less, such as from about 10 to about 200 ms, such as from about 15 to about 20 ms.

In an embodiment the exciting RF pulse or train of pulses has a field band width (Hz), a pulse width (μs) and amplitude (Volt) selected to provide the desired angle pulse, such as a 45° pulse, a 90° or a 180° pulse, preferably the field band width of the pulse up to about 1 KHz, such as from about 100 to about 500 Hz, such as from about 150 to about 300 Hz.

The phrase “an X° pulse” where X can be any degree should be interpreted to include a train of X° pulses unless otherwise specified.

In an embodiment the NMR measurement comprises simultaneously subjecting the sample to a magnetic field B, and a plurality of RF pulses wherein the RF pulses comprise a plurality of exciting RF pulses and a plurality of refocusing RF pulses.

Advantageously the exciting RF pulses are soft pulses having field band width of up to about 1 KHz, such as from about 100 to about 500 Hz, such as from about 150 to about 300 Hz.

In principle the refocusing RF pulses may have any have any field band width and often it is desired to apply refocusing RF pulses with a relatively high field band width in order to reduce the pulse width.

In an embodiment the method of the invention comprises determining at least one relaxation rate of the exited nuclei in the retentate fraction.

Methods of measuring relaxation times T1 and T2 are well known in the art.

In an embodiment the method comprises subjecting the retentate fraction to pulsed trains of RF pulses, preferably with repetition rates of at about 100 ms or less, such as from about 10 to about 50 ms, such as from about 15 to about 20 ms.

The trains of RF pulses may for example be applied to determine the T1 and/or T2 values.

A short square pulse of a given “carrier” frequency “contains” a range of frequencies centered about the carrier frequency, with the range of excitation (bandwidth/frequency spectrum) being inversely proportional to the pulse duration.

A Fourier transform of an approximately square wave contains contributions from all the frequencies in the neighborhood of the principal frequency. The restricted range of the NMR frequencies made it relatively easy to use short (millisecond to microsecond) radio frequency pulses to excite the entire NMR spectrum.

In an embodiment the NMR measurement comprises simultaneously subjecting the sample to a magnetic field B and a plurality of RF pulses wherein the RF pulses comprise
i. an exciting RF pulse, and
ii. at least one refocusing RF pulse.

The exciting RF pulse and the refocusing pulse or pulses may for example be in the form of a train of RF pulses, e.g. pulsed pulses. The exciting RF pulse is preferably as described above and may in an embodiment be pulsed.

Useful duration and amplitude of the exciting RF pulses are well known in the art and optimization can be done by a simple trial and error.

In an embodiment the exciting RF pulse is in the form of a 90° pulse.

A 90° pulse is an RF pulse designed to rotate the net magnetization vector 90° from its initial direction in the rotating frame of reference. If the spins are initially aligned with the static magnetic field, this pulse produces transverse magnetization and free induction decay (FID).

In an embodiment the refocusing RF pulse(s) is in the form of a 180° pulse, preferably the method comprises subjecting the sample to a plurality of refocusing RF pulses, such as one or more trains of refocusing RF pulses.

A 90° pulse is an RF pulse designed to rotate the net magnetization vector 180° in the rotating frame of reference. Ideally, the amplitude of a 180° pulse multiplied by its duration is twice the amplitude of a 90° pulse multiplied by its duration. Each 180° pulse in the sequence (called a CPMG sequence after Carr-Purcell-Meiboom-Gill) creates an echo.

A standard technique for measuring the spin-spin relaxation time T2 utilizing CPMG sequence is as follows. As is well known after a wait time that precedes each pulse sequence, a 90-degree exciting pulse is emitted by an RF antenna, which causes the spins to start processing in the transverse plane. After a delay, an initial 180-degree pulse is emitted by the RF antenna. The initial 180-degree pulse causes the spins, which are dephasing in the transverse plane, to reverse direction and to refocus and subsequently cause an initial spin echo to appear. A second 180-degree refocusing pulse can be emitted by the RF antenna, which subsequently causes a second spin echo to appear. Thereafter, the RF antenna emits a series of 180-degree pulses separated by a short time delay. This series of 180-degree pulses repeatedly reverse the spins, causing a series of “spin echoes” to appear. The train of spin echoes is measured and processed to determine the spin-spin relaxation time T2.

In an embodiment the refocusing RF pulse(s) is/are applied with an echo-delay time after the exciting RF pulse. The echo-delay time (also called wait time TW) is preferably of about 500 μs or less, more preferably about 150 μs or less, such as in the range from about 50 μs to about 100 μs.

This method is generally called the “spin echo” method and was first described by Erwin Hahn in 1950. Further information can be found in Hahn, E. L. (1950). “Spin echoes”. Physical Review 80: 580-594, which is hereby incorporated by reference.

A typical echo-delay time is from about 10 μs to about 50 ms, preferably from about 50 μs to about 200 μs. The echo-delay time (also called wait time TW) is the time between the last CPMG 180° pulse and the first CPMG pulse of the next experiment at the same frequency. This time is the time during which magnetic polarization or T1 recovery takes place. It is also known as polarization time.

This basic spin echo method provides very good result for obtaining T1 relaxation values by varying TW and T2 relaxation values can also be obtained by using plurality of refocusing pulses.

The refocusing delay is also called the Echo Spacing and indicates the time identical to the time between adjacent echoes. In a CPMG sequence, the TE is also the time between 180° pulses.

This method is an improvement of the spin echo method by Hahn. This method was provided by Carr and Purcell and provides an improved determination of the T2 relaxation values which again allows for better quantitative determination of the isotope(s) via more precise elimination of T2 effects via single or multi curve fitting for most precise envelope of spin echo amplitudes.

Further information about the Carr and Purcell method can be found in Carr, H. Y.; Purcell, E. M. (1954). “Effects of Diffusion on Free Precession in Nuclear Magnetic Resonance Experiments”. Physical Review 94: 630-638, which is hereby incorporated by reference.

In an embodiment the NMR measurement comprises subjecting the sample to proton decoupling pulses and/or polarization pulses during at least a part of the NMR reading. This method has been found to increase the accuracy of the resulting isotope/compound determination.

In an embodiment the method comprising enhancing signal to noise of the data spectra by subjecting the sample to a pulse configuration providing a polarization and/or a proton decoupling of atoms one or more compounds in the sample.

In an embodiment the method comprising enhancing signal to noise of the data spectra by subjecting the sample to a pulse configuration comprising at least one of DEPT (Distortionless Enhancement by Polarization Transfer), DEPTQ (DEPT with retention of Quaternaries), HSQC (Heteronuclear Single Quantum Coherence), INEPT (Insensitive Nuclei Enhanced by Polarization Transfer), BIRD (Bilinear Rotation Decoupling pulses), TANGO (Testing for Adjacent Nuclei with a Gyration Operator) or NOE (Nuclear Overhauser Effect). Further information about these pulse configurations can be found in co-pending patent application DK-PA-2014 70339.

In an embodiment the method comprises determine a quality parameter based at least partly on a quantitative determination on ¹⁷O determined as described in co-pending patent application DK-PA-2014 70339 with the difference that the aqueous fluid sample has been subjected to a cross-flow filtration and the NMR reading is performed on the retentate fraction.

The invention also relates to a method of controlling a quality parameter of an aqueous fluid. The method comprises determine the quality parameter using the method as described above and comparing the determined quality parameter to a set point range for the quality parameter and if the determined quality parameter is not within the set point range for the quality parameter, treating the aqueous fluid by adding and/or withdrawing component(s) from the aqueous fluid or by modifying an addition/withdrawing treatment of the aqueous fluid.

In an embodiment the quality parameter comprises nitrogen content, flour content, chlorine content, content of free chlorine (HOCL, OCl⁻), content of ammonium, content of ammonia, content of nitrate, content of nitrite, content of potassium, content of phosphor, content of organic matter, content of organic solvents, such as benzene, content of heavy metal(s), content of trihalomethane, content of total carbons (TC), content of total organic carbon (TOC), content of selected hydrocarbons (e.g. methane or butane), or any combinations thereof.

In an embodiment the aqueous fluid is drinking water, waste water, industrial waste water, municipal waste water, lake water, sea water, swimming pool water, aquaculture water or laboratory water sample.

The invention also relates to a NMR system suitable for determining a quality parameter in an aqueous fluid. The NMR system comprises a NMR spectrometer, a cross-flow filter, a digital memory storing a calibration map comprising calibrating data for calibrating NMR data obtained by the NMR spectrometer and a computer programmed to analyze the NMR data obtained by the NMR spectrometer using the calibration map and performing at least one quantitative and/or qualitative quality parameter determination.

Preferably the cross-flow filter is configured for subjecting at least a sample of the aqueous fluid to a cross-flow filtration to separate the separating the aqueous fluid sample into a permeate fraction and a retentate fraction.

The cross-flow filtration advantageously is as described above.

Preferably the NMR spectrometer is configured for performing NMR reading on the retentate fraction. Advantageously the NMR spectrometer is as described.

Preferably the computer is configured for collecting NMR data from the NMR reading and correlating the collected NMR data to calibration data to determine the at least one quality parameter of the aqueous fluid.

Advantageously the NMR system is configured for performing the method as described above.

The computer may be a single computer or it may comprise a plurality of sub-computers in data communication with each other.

The digital memory may be incorporated in the computer or it may be an external data unit e.g. accessible via the internet.

In an embodiment at least the NMR spectrometer and the cross-flow filter is arranged in a common housing. It has been found that the common housing comprising the NMR spectrometer and the cross-flow filter can be a very compact module as it will be described further in the examples.

In an embodiment the cross-flow filter, the NMR spectrometer and the computer filter in arranged in the common housing

Advantageously the cross-flow filter is a multi stage cross-flow filter comprising at least two filter membranes, the two filter membranes may be equal or different and may be operating with same or different pressure difference over the respective filter membranes.

In an embodiment the cross-flow filter is an exchangeable cross-flow filter, preferably arranged for manually removal and replacement by an operator. Thereby the NMR system can be used for different aqueous fluid with different concentrations and/or type of impurities.

In an embodiment the NKR system comprises a pre-filter unit arranged to pre-filter the aqueous fluid sample to remove at least some solids prior to subjecting the sample to the cross-flow filtration, optionally the removed solids is subjected to NMR readings e.g. after being mixed with the retentate fraction.

All features of the inventions including ranges and preferred ranges can be combined in various ways within the scope of the invention, unless there are specific reasons not to combine such features.

### BRIEF DESCRIPTION OF EXAMPLES AND DRAWINGS

The invention is being illustrated further below in connection with a few examples and embodiment and with reference to the drawings in which:

Table 1 shows a number of examples of quality parameters which may be determined according to the invention.

Table 2 shows examples of quality guidelines for drinking water.

FIG. 1 shows an example of an NMR system of the invention.

FIG. 2 shows another example of an NMR system of the invention.

FIG. 3 shows a further example of an NMR system of the invention.

The figures are schematic and simplified for clarity. As it will be clear to the skilled person the systems illustrated may comprise more or less units, such as more or less pumps, valves and similar which will be within the ordinary skill of a person skilled in the art to modify within the scope of the invention.

Table 1 lists number of selected quality parameters which may be determined according to the invention. It is indicated on which isotopes the determination can be based and there are also provided examples of suitable types of aqueous fluid for which the respective quality parameter could be useful. It should be understood that the list is in no way exhaustive and that numerous other quality parameter could be determined using the method of the invention.

Table 2 shows examples of quality guidelines for drinking water with focus of the maximal recommended levels of a number of heavy metals. As it can be seen the levels are very low and are often difficult to measure with any desired precision using prior art methods. By use of the method of the invention the amount of the respective heavy metal in mg/I or even sub mg/I level can be determined with a high accuracy.

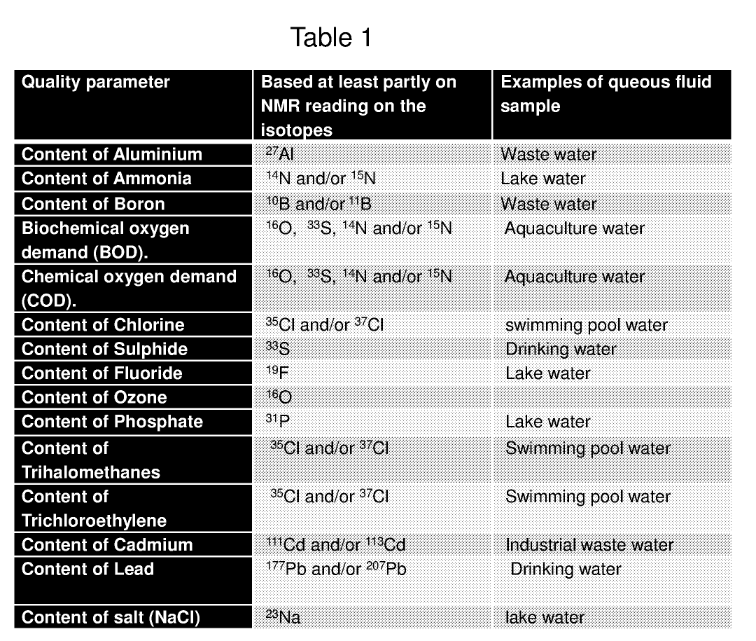

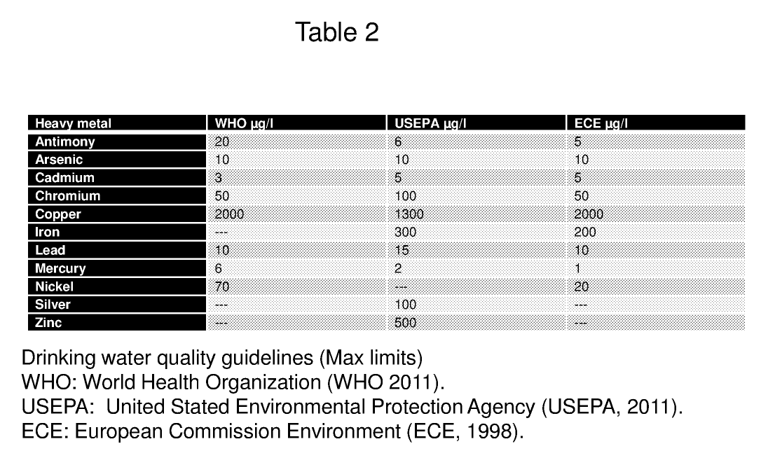

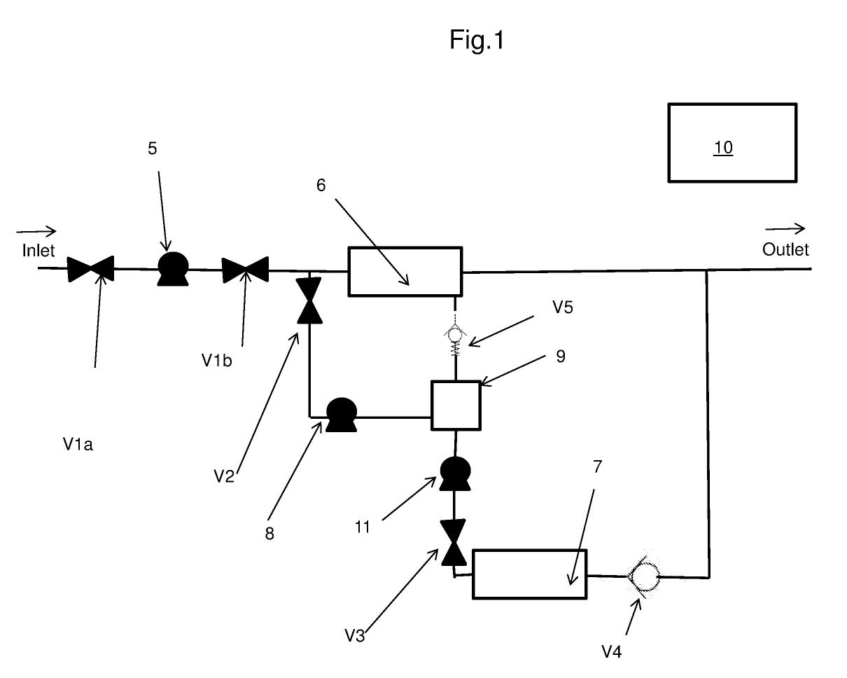

The NMR system shown in FIG. 1 comprises a NMR spectrometer 7, a cross-flow filter 6 and a computer 10 comprising a digital memory storing a calibration map comprising calibrating data for calibrating NMR data obtained by the NMR spectrometer.

The NMR system comprises an inlet and an outlet as marked as well as a number of valves V1a, V1b, V2, V3, a one way valve V4, a spring valve V5, a retentate fraction reservoir 9 and three pumps 5, 8 and 11. The computer 10, is digital connected with not shown connection to control the system and to obtain the NMR data from the NMR spectrometer 7.

In use the aqueous fluid sample is fed to the system via the inlet. The valves V1a and V1b are open and valves V2 and V3 are closed. The spring valve V5 ensures a desired overpressure in the cross-flow filter 6 to ensure a pressure over the membrane of the cross-flow filter 6. The aqueous fluid sample is pumped by pump 5 through valves V1a and V1b and into the cross-flow filter 6. The permeate fraction is let to the outlet and the retentate fraction is let to the retentate fraction reservoir 9.

When the whole aqueous fluid sample has been fed into the NMR system, the pump 5 is shut off, valves V1a and V1b are closed, valves V2 is opened. The pump 8 is now started and the retentate fraction will be recirculated through the cross-flow filter 6. The pressure over the cross-flow filter is regulated by the pump 8 and the spring valve V5. This recirculation may be continued for a time e.g. as described above. Thereafter the valve V2 is closed and the Valves V3 is opened. The pump 11 is set to pump the retentate fraction from the retentate fraction reservoir 9 into the NMR spectrometer 7. The NMR system may be arranged to perform NMR readings on a portion of the retentate fraction at a time when the pump is stopped, the NMR reading is performed and the retentate fraction portion is pumped out via valve V4 which is opened by the pump pressure for discharging the portion. Alternatively the pump will pump with a relatively low power to ensure a low velocity of the retentate fraction and valve V4 remains open and the NMR reading is performed on the retentate fraction in flow through the NMR spectrometer 7.

The obtained NMR data is transmitted to the computer for processing e.g. as described above to determine at least one quality parameter.

Advantageously the NMR spectrometer and the cross-flow filter and optionally the computer are arranged in a not shown common housing.

The cross-flow filter is advantageously as described above.

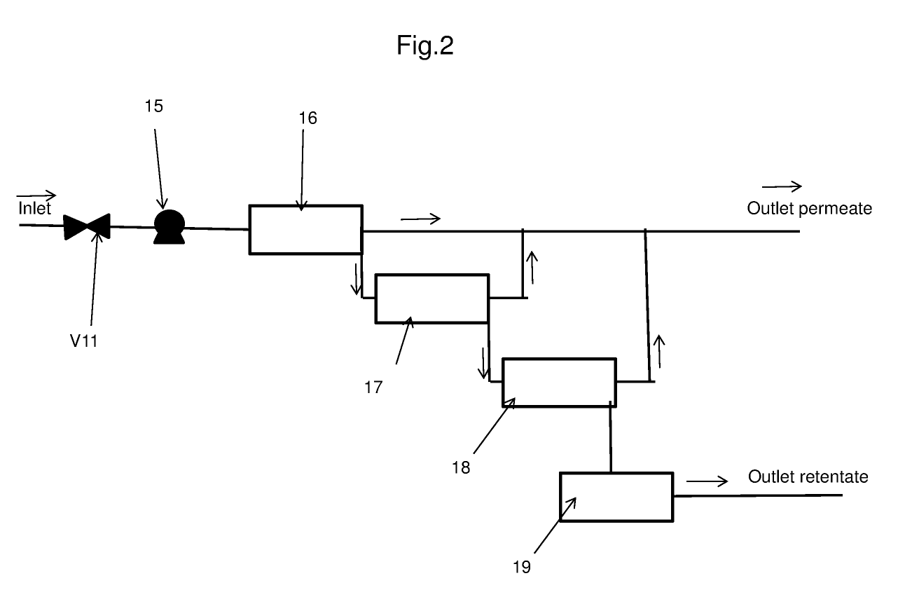

The NMR system shown in FIG. 2 comprises a NMR spectrometer 19, a cross-flow filter comprising a number of separate filter membranes 16, 17, 18 and a not shown computer in data communication with a digital memory storing a calibration map comprising calibrating data for calibrating NMR data obtained by the NMR spectrometer.

The NMR system comprises an inlet and an outlet for permeate and an outlet for retentate. The NMR system further comprises at least one valve V11 and at least one pump 15. The NMR system advantageously comprises one or more not shown spring valves to ensure a desired pressure over the respective filter membranes 16, 17, 18.

The computer is connected with not shown connection to control the system and to obtain the NMR data from the NMR spectrometer 19.

In use the aqueous fluid sample is fed to the system via the inlet. The valve V1 is open and the pump 15 is turned on. The aqueous fluid sample is pumped into the first filter membrane 16. The permeate fraction is let to the permeate outlet and the retentate fraction is let to the 2^{nd} filter membrane 17. After having passed the 2^{nd} filter membrane 17, the permeate fraction is let to the permeate outlet and the retentate fraction is let to the 3^{rd} filter membrane filter 18. After having passed the 3^{rd} filter membrane 18, the permeate fraction is let to the permeate outlet and the retentate fraction is let to NMR spectrometer 19 where it is subjected to the NMR reading as described above.

As it can be seen the number of filter membrane cross-flow filter in such cascade design cross-flow filter can in a simple way be regulated and the individual cross-flow filter membrane s can be identical or different from eat other. If desired one or more additional pumps can be applied to regulate the pressure over the respective cross-flow filter membrane 16, 17, 18. The pressure over the respective cross-flow filter membrane 16, 17,18 may be equal or different from each other and the filter membranes 16, 17, 18 may as well be equal or different from each other.

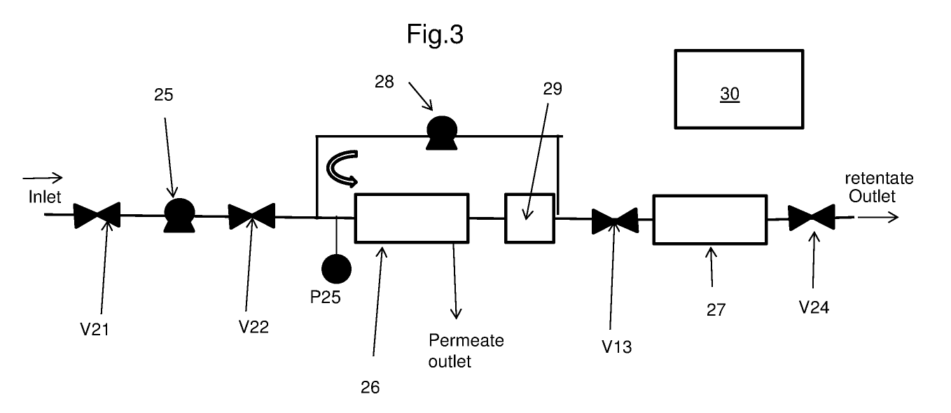

The NMR system shown in FIG. 3 comprises a NMR spectrometer 27, a cross-flow filter 26 and a computer 30 comprising a digital memory storing a calibration map comprising calibrating data for calibrating NMR data obtained by the NMR spectrometer.

The NMR system comprises an inlet, a permeate outlet and a retentate outlet as marked. The system also comprises a number of valves V21a, V21b, V22, V23, a one way valve V24, a pressure control unit P25, an optional retentate fraction reservoir 29 and two pumps 25, 28. The computer 30 is digital connected with not shown connection to control the system and to obtain the NMR data from the NMR spectrometer 27.

The pump 25 ensures a suitable pressurization of the RO loop and the pressure control unit P25 is used for pressure control. The Pump 25 may advantageously be a volumetric piston pump (allows calculation of concentration factor) or alternatively a non-volumetric pump. In the latter case it is desired to measure (e.g. volume or concentration of at least one isotope) before and after RO-loop to determine the concentration factor.

The cross-flow filter 26 is a reverse osmosis unit. The pump 28 is a circulation pump. Advantageously the total inner volume of the cross-flow filter 26, the optional retentate fraction reservoir 29, the pumps 25, 28 and the connecting pipes may be relatively small e.g. smaller than 1 L. In an embodiment the NMR needs only 10 mL or less.

In use the aqueous fluid sample is fed to the system via the inlet.

The complete system, i.e. the cross-filtration loop and the pipe through the NMR is filled with the aqueous fluid using pump 5 while valves V21, V22, V23 and V24 are open.

To enrich/concentrate the aqueous fluid, valves V23 and V24 are closed and pump 25 continues to pump aqueous fluid into the cross-filtration loop thereby increasing the pressure inside the loop. Pressure control unit P25 may be arranged to control pump 25 to keep the pressure within a preset range. Upon increasing the pressure inside the cross-filtration filter 26, a permeate is pressed through the filter and is discarded via the permeate outlet. Pump 28 ensures maintaining a sufficient high fluid flow across the membrane of the cross-filtration filter 26 to minimize membrane fouling. Given the internal fluid volume of the loop (cross-filtration filter 26, pump 28, optional retentate fraction reservoir 29 and connecting pipes connecting the units), additional aqueous fluid pushed into the loop will lead to an increase of aqueous fluid sample and thereby the total amount of the isotope(s) of interest in the final retentate fraction. Pushing e.g. 9 L of additional aqueous fluid into a loop with a volume of 1 L leads to an enrichment of a factor of 10. To calculate the enrichment factor, pump 25 is preferably of a volumetric type (e.g. piston pump).

When the enrichment is finished, valve V23 and V24 are opened and pump 25 is used to transport the enriched aqueous fluid into the NMR unit for analysis. When the NMR analysis is finished, a new cycle is started by flushing the complete system with aqueous fluid through the inlet and out via the retentate outlet.

The enrichment/concentration factor may also be calculated by comparing the concentration of an isotope or a compound comprising an isotope in the original aqueous fluid (unfiltered) at the startup of the system with the concentration of the species in the enriched fluid (the retentate fraction) at the final NMR analysis.

### EXAMPLES

### Example 1

5000 ml sample of water from a swimming pool is obtained. The sample is fed to a NMR system as shown in FIG. 1.

The cross-flow filter membrane is of RO type, for example of the Axeon HR3 Series Reverse Osmosis Membranes marketed by Fresh Water Systems Inc. Greenville, S.C.

The pressure over the cross-flow filter is 10 bars.

The sample is recirculated through the cross-flow filter for 30 minutes. The resulting volume of the retentate fraction: 200 ml.

A portion of 100 ml of the retentate fraction is led into the NMR spectrometer for ³⁵Cl NMR reading.

The test in the NMR spectrometer is performed at a substantially homogeneous field of about 1.5 Tesla. The ³⁵NMR reading comprises reading of T1 and T2 data, data obtained by DEPT and/or NOE. The accumulated NMR reading time is 30 minutes.

The obtained NMR data is transmitted to the computer for calibrating with a calibration map comprising ³⁵Cl NMR data obtained from swimming pool water samples with known amounts.

The computer is programmed to determine the chlorine content of the swimming pool water based on the obtained NMR data.

### Example 2

1000 ml sample of surface water from a lake is obtained and supplied for analysis. The sample is fed to a NMR system as shown in FIG. 1.

The cross-flow filter membrane is of RO type

The pressure over the cross-flow filter is 10 bars.

The sample is recirculated through the cross-flow filter for 5 minutes.

The resulting volume of the retentate fraction: 100 ml

A portion of 50 ml of the retentate fraction is led into the NMR spectrometer for ¹⁴N NMR reading.

The test in the NMR spectrometer is performed at a substantially homogeneous field of about 1.5 Tesla. The ¹⁴N and NMR reading comprises reading of T1 and T2 data, data obtained by DEPT and/or NOE. Further ³¹P and ³⁹K NMR data was obtained. The accumulated NMR reading time is 5 minutes.

The obtained NMR data is transmitted to the computer for calibrating with a calibration map comprising ¹⁴N, ³¹P, ³⁹K NMR data obtained from lake water samples with known amounts.

The computer is programmed to determine the NPK quality parameter of the lake water based on the obtained NMR data.

### Example 3

1000 liter is sample of drinking water from is obtained. The sample is fed to a NMR system as shown in FIG. 2.

The 3 cross-flow filter membranes were of UF, NF and finally RO type. The pressure over each of the cross-flow filter is 5 bars.

The resulting volume of the retentate fraction: 100 ml

A portion of 50 ml of the retentate fraction is led into the NMR spectrometer for ²⁰⁷PB and ⁶³Cu NMR reading.

The test in the NMR spectrometer is performed at a substantially homogeneous field of about 1.5 Tesla. The ²⁰⁷PB and ⁶³Cu NMR reading comprises reading of T1 and T2 data, data obtained by DEPT and/or NOE. The accumulated NMR reading time is 24 hours.

The obtained NMR data is transmitted to the computer for calibrating with a calibration map comprising ²⁰⁷PB and ⁶³Cu NMR data obtained from drinking water samples with known amounts.

The computer is programmed to determine the amount of lead in the drinking water based on the obtained NMR data.

Further scope of applicability of the present invention will become apparent from the detailed description given herein. However, it should be understood that the detailed description and specific examples, while indicating preferred embodiments of the invention, are given by way of illustration only, since various changes and modifications within the spirit and scope of the invention will become apparent to those skilled in the art from this detailed description.

What is claimed is:

1-59. (canceled)

60. A method of determining a least one quality parameter in an aqueous fluid, the method comprising subjecting at least a sample of the aqueous fluid to a cross-flow filtration in a cross-flow filter, separating the aqueous fluid into a permeate fraction and a retentate fraction, performing NMR reading on the retentate fraction using an NMR spectroscope, collecting NMR data from said NMR reading and correlating the collected NMR data to calibration data to determine said at least one quality parameter of the aqueous fluid.

61. The method of claim 60, wherein the cross-flow filtration is adjusted such that the permeate fraction is larger than the retentate fraction.

62. The method of claim 60, wherein the method comprises determining the relative mass or volume of said retentate fraction relative to mass or volume of at least one of the sample or the permeate.

63. The method of claim 60, wherein the method comprises recirculating the retentate fraction in the cross-flow filter followed by performing NMR reading on the retentate fraction.

64. The method of claim 60, wherein the at least one NMR reading comprises a reading at least one NMR readable isotope selected from the isotopes ¹H, ¹⁰B, ¹¹B, ¹³C, ¹⁴N, ¹⁵N, ¹⁶O, ¹⁹F ²³Na, ²⁷Al, ²⁹Si ³¹P, ³³S, ³⁵Cl, ³⁷Cl, and ³⁹K, ⁴¹K, ⁴³Ca, ⁴⁷Ti, ⁴⁹Ti, ⁵⁰V, ⁵¹V, ⁵³Cr, ⁵⁵Mn, ⁵⁷Fe, ⁵⁹Co, ⁶¹Ni, ⁶³Cu, ⁶⁵Cu, ⁶⁷Zn, ⁶⁹Ga, ⁷¹Ga, ⁷⁵As, ⁷⁷Se, ⁷⁹Br, ⁸¹Br, ⁸³Kr, ⁸⁵Rb, ⁸⁷Rb, ⁸⁷Sr, ⁸⁹Y, ⁹¹Zr, ⁹³Nb, ⁹⁵Mo, ⁹⁷Mo, ¹⁰⁵Pd, ¹⁰⁷Ag, ¹⁰⁹Ag, ¹¹¹Cd, ¹¹³Cd, ¹¹⁷Sn, ¹¹⁹Sn, ¹¹⁵Sn, ¹²¹Sb, ¹³⁵Ba, ¹³⁷Ba ¹⁷⁷Pb, ¹⁹⁹Hg, ²⁰¹Hg, ²⁰⁷Pb.

65. The method of claim 64, wherein the method comprises NMR reading of one or more heavy metal isotopes, such as isotopes of Pb, Hg and/or Cd.

66. The method of claim 64, wherein the method comprises NMR reading of ³⁵Cl and/or ³⁷Cl and qualitatively and/or quantitatively determine one or more trihalomethanes and/or free chlorine and/or total chlorine contents.

67. The method of claim 60, wherein the at least one quality parameter comprises nitrogen content, flour content, chlorine content, content of free chlorine (HOCL, Off), content of ammonium, content of ammonia, content of nitrate, content of nitrite, content of potassium, content of phosphor, content of organic matter, content of organic solvents, such as benzene, content of heavy metal(s), content of trihalomethane, content of total carbons (TC), content of total organic carbon (TOC), content of selected hydrocarbons (e.g. methane or butane), or any combinations thereof.

68. The method of claim 60, wherein the method comprises providing a control loop adjusting the cross-flow filtration such that to obtain a preselected flux through the cross-flow filter to become permeate, wherein the preselected percentage is from about 50 to about 99 vol %, such as from about 60 to about 95 vol %.

69. The method of claim 60, wherein the method comprises performing NMR reading on an unfiltered sample of the aqueous fluid, preferably the NMR reading on the unfiltered sample comprises NMR reading of at least one isotope which is also read on the retentate fraction.

70. The method of claim 60, wherein the method comprises calibrating the cross-flow filtration performance based on the difference in NMR data of the retentate fraction NMR reading and NMR data of the unfiltered sample NMR reading.

71. The method of claim 60, wherein the NMR reading comprises subjecting the retentate fraction to proton decoupling pulses and/or polarization pulses during at least a part of the NMR reading.

72. The method of claim 60, wherein the NMR reading comprises enhancing signal to noise of the data spectra by subjecting the retentate fraction to a pulse configuration comprising at least one of DEPT (Distortionless Enhancement by Polarization Transfer), DEPTQ (DEPT with retention of Quaternaries), HSQC (Heteronuclear Single Quantum Coherence), INEPT (Insensitive Nuclei Enhanced by Polarization Transfer), BIRD (Bilinear Rotation Decoupling pulses), TANGO (Testing for Adjacent Nuclei with a Gyration Operator) or NOE (Nuclear Overhauser Effect).

73. A method of controlling a quality parameter of an aqueous fluid, the method comprises determine the quality parameter using the method of claim 60, comparing the determined quality parameter to a set point range for the quality parameter and if the determined quality parameter is not within the set point range for the quality parameter, treating the aqueous fluid by adding and/or withdrawing component(s) from the aqueous fluid or by modifying an addition/withdrawing treatment of the aqueous fluid.

74. The method of controlling a quality parameter of claim 73, wherein the quality parameter comprises nitrogen content, flour content, chlorine content, content of free chlorine (HOCL, OCl⁻), content of ammonium, content of ammonia, content of nitrate, content of nitrite, content of potassium, content of phosphor, content of organic matter, content of organic solvents, such as benzene, content of heavy metal(s), content of trihalomethane, content of total carbons (TC), content of total organic carbon (TOC), content of selected hydrocarbons (e.g. methane or butane), or any combinations thereof.

75. A NMR system suitable for determining a quality parameter in an aqueous fluid, the system comprises a NMR spectrometer, a cross-flow filter, a digital memory storing a calibration map comprising calibrating data for calibrating NMR data obtained by the NMR spectrometer and a computer programmed to analyze the NMR data obtained by the NMR spectrometer using the calibration map and performing at least one quantitative and/or qualitative quality parameter determination.

76. The NMR system of claim 75, wherein the cross-flow filter is configured for subjecting at least a sample of the aqueous fluid to a cross-flow filtration to separate the separating the aqueous fluid sample into a permeate fraction and a retentate fraction, the NMR spectrometer is configured for performing NMR reading on the retentate fraction and the computer is configured for collecting NMR data from said NMR reading and correlating the collected NMR data to calibration data to determine said at least one quality parameter of the aqueous fluid.

77. The NMR system of claim 75, wherein at least the NMR spectrometer and the cross-flow filter are arranged in a common housing.

78. The NMR system of claim 75, wherein the cross-flow filter comprises a ceramic filter membrane, a thin-film composite membrane (TFC) and/or a polymer membrane.

79. The NMR system of claim 75, wherein the cross-flow filter comprises a flat sheet membrane and/or a coiled membrane (spiral membrane)

80. The NMR system of claim 75, wherein the cross-flow filter is a reverse osmosis filter and the cross-flow filtration is reverse osmosis.
